SEQUENCE LISTING (27 sequences)

SEQ ID NO: 1 - DNA, 275 bp, Ageratum yellow vein virus
aagagattctgtgtgaagtctgtttatgtgttaggtaaaatatggatggatgaaaatattaaaactaagaaccatacgaacactgtgatgttttttcttgttcgtgacagaaggccctatggtactgctatggattttggtcaggtgtttaacatgtatgataatgagcccagtactgctactatcaagaatgatcttcgagatcgttatcaagttttaaggaaattcagttcaacagtcacagggggtcaatatgcttctaaggaacaggcgtt

SEQ ID NO: 2 - DNA, 147 bp, Ageratum yellow vein virus
tatggattttggtcaggtgtttaacatgtatgataatgagcccagtactgctactatcaagaatgatcttcgagatcgttatcaagttttaaggaaattcagttcaacagtcacagggggtcaatatgcttctaaggaacaggcgtt

SEQ ID NO: 3 - DNA, 126 bp, Ageratum yellow vein virus
taacatgtatgataatgagcccagtactgctactatcaagaatgatcttcgagatcgttatcaagttttaaggaaattcagttcaacagtcacagggggtcaatatgcttctaaggaacaggcgtt

SEQ ID NO: 4 - DNA, 105 bp, Ageratum yellow vein virus
cagtactgctactatcaagaatgatcttcgagatcgttatcaagttttaaggaaattcagttcaacagtcacagggggtcaatatgcttctaaggaacaggcgtt

SEQ ID NO: 5 - DNA, 108 bp, Ageratum yellow vein virus
taacatgtatgataatgagcccagtactgctactatcaagaatgatcttcgagatcgttatcaagttttaaggaaattcagttcaacagtcacagggggtcaatatgc

SEQ ID NO: 6 - DNA, 23 bp, artificial
  synthetic PCR primer
ccatatctatatctccttnnnnn

SEQ ID NO: 7 - DNA, 20 bp, artificial
  synthetic PCR primer
taacatgtatgataatgagc

SEQ ID NO: 8 - DNA, 20 bp, artificial
  synthetic PCR primer
ccagtactgctactatcaag

SEQ ID NO: 9 - DNA, 21 bp, artificial
  synthetic PCR primer
actatcaagatgatcttcgag

SEQ ID NO: 10 - DNA, 22 bp, artificial
  synthetic PCR primer
aagcttaagtttaaacgctagc

SEQ ID NO: 11 - DNA, 17 bp, artificial
  synthetic PCR primer
tatggattttggtcagg

SEQ ID NO: 12 - DNA, 20 bp, artificial
  synthetic PCR primer
gtaagcttgcatattgaccc

SEQ ID NO: 13 - DNA, 20 bp, artificial
  synthetic PCR primer
gcaagctttatggattttgg

SEQ ID NO: 14 - DNA, 20 bp, artificial
  synthetic PCR primer
taggatccgcatattgaccc

SEQ ID NO: 15 - DNA, 20 bp, artificial
  synthetic PCR primer
gcggatcctatggattttgg

SEQ ID NO: 16 - DNA, 30 bp, artificial
  synthetic PCR primer
ccatatctatatctccttgcatattgaccc

SEQ ID NO: 17 - DNA, 17 bp, artificial
  synthetic PCR primer
gcatattgacccccctg

SEQ ID NO: 18 - DNA, 32 bp, artificial
  synthetic PCR primer
ccatatctatatctcctttatcgattttggtc

SEQ ID NO: 19 - DNA, 19 bp, artificial
  synthetic PCR primer
aagggcaattctgcagatc

SEQ ID NO: 20 - DNA, 19 bp, artificial
  synthetic PCR primer
taaaacttgataacgatct

SEQ ID NO: 21 - DNA, 15 bp, artificial
  synthetic PCR primer
gcaaacaacagatgg

SEQ ID NO: 22 - DNA, 21 bp, artificial
  synthetic PCR primer
aattgcgagcacgaattactg

SEQ ID NO: 23 - DNA, 36 bp, artificial
  synthetic PCR primer
ccatatctatatctcctttttgactaagtcaattgg

SEQ ID NO: 24 - DNA, 36 bp, artificial
  synthetic PCR primer
cctcttgtcaggccggaataactccctataatgcgc

SEQ ID NO: 25 - DNA, 36 bp, artificial
  synthetic PCR primer
ttatccgctcacaattccctggtggcgcattatagg

SEQ ID NO: 26 - DNA, 37 bp, artificial
  synthetic PCR primer
aacaaaattatttctagaggggaattgttatccgctc

SEQ ID NO: 27 - DNA, 39 bp, artificial
  synthetic PCR primer
ccatatctatatctccttttaaagttaaacaaaattatt

(19) United States
(12) Patent Application Publication
(10) Pub. No.: US 2011/0053258 A1
(43) Pub. Date: Mar. 3, 2011

(54) Novel promoter sequence and the application thereof

(76) Inventors: Chung-Chi Hu, Taichung City (TW); Wei-Chen Wang, Taichung City (TW); Chia-Ying Wu, Taichung City (TW); Yau-Heiu Hsu, Taichung City (TW); Yi-Chin Lai, Taichung City (TW)

(21) Appl. No.: 12/155,393

(22) Filed: Jun. 3, 2008

(51) Int. Cl. C12N 15/63; C07H 21/00

(52) U.S. Cl. 435/320.1; 536/24.1

(57) ABSTRACT

The present invention provides a novel promoter sequence derived from a promoter of geminivirus, a eukaryotic virus, and has the characteristics of prokaryotic promoters. The isolated sequence includes SEQ ID NO: 5, which can drive the expression of foreign genes in prokaryotic cells to a high level utilizing the prokaryotic RNA polymerases. The present invention is a constitutive promoter which does not require the addition of external inducers to promote high-level gene expressions. The promoter activity of the present invention is 15-fold higher than the Rep gene promoter of geminivirus, which is also active in prokaryotic cells. Compared to the promoter activity of the standard constitutive prokaryotic promoter rrnB P1, the activity of the present invention is 11.1% higher. The activity of the present invention is additive when concatenated in the same polarity in the constructs, further enhancing the expression level of genes.

### BACKGROUND OF THE INVENTION

1. Field of the Invention

The present invention relates to a novel promoter sequence and its application thereof.

2. Description of the Related Art

Geminiviruses, with mono- or bi-partite genomes, infect plants, and therefore belong to eukaryotic systems. Since geminiviruses do not encode DNA polymerases, their DNA replication cycle relies largely on the use of eukaryotic cellular DNA replication proteins. However, the strategy used by geminiviruses to replicate their single-stranded DNA (ssDNA) genome is by a rolling-circle replication mechanism. Such a strategy is the same as that used for replication by some bacterial phages, which belong to prokaryotic systems. Further, according to previous studies, geminiviruses could also replicate in prokaryotic cells, for example, (1) their DNA replication could be supported in Agrobacterium umefaciens and E. coli: Rigden et al. (1996), transformed an isolated plasmid harboring genomic sequences of Tomato leaf curl virus (TLCV) into A. umefaciens and found the accumulation of single-stranded circular genomic DNA of TLCV. The above replication of TLCV genomic DNA occurred only when C1 gene of TLCV expressed functional Rep protein and the plasmid construct contained two copies of replication origin (Ori) sequences. Later, by using similar strategy for plasmid constructions, Selth et al., (2002) also demonstrated that Tomato yellow leaf curl virus (TYLCV) and African cassaya mosaic virus (ACMV) were both replicated in A. umefaciens. In addition, low-level replication of TLCV DNAs were also detected in Escherichia coli. By replacing 6 known genes (Rep, TrAP, Ren, AC4, AV1, CP) of geminiviruses with β-glucuronidase reporter gene, Selth et al. showed that all the 6 promoters were activated in A. umefaciens and two of them were also functional in E. coli, although the expression levels were relatively low. Furthermore, the applicants (Wu et al., 2007) has demonstrated that unit-length, single-stranded circular DNAs of both polarity of Agreratum yellow vein virus (AYVV) could be generated in E. coli harboring phage M13-cloned AYVV genome with a single copy of Ori.

### SUMMARY OF THE INVENTION

Accordingly, the previous studies indicated that eukaryotic geminiviruses possess the ability to express their genes and replicate in the prokaryotic systems, and raised the possibility that geminiviruses might encode other unknown regulatory sequences or genes that confer the abilities of geminiviruses to thrive in both systems. If present, such regulatory sequences or genes would be of significant values in the fields of molecular virology and biotechnology. The applicants have endeavored to search for previously unknown regulatory sequences or genes in AYVV genome as a model system, and identified a novel promoter sequence that is highly active in prokaryotic systems.

Therefore, the primary objective of the present invention is to provide an isolated promoter sequence comprising SEQ ID NO: 5 or a substantial identity sequence.

Preferably, SEQ ID NO: 5 is isolated from Ageratum yellow vein virus.

More preferably, SEQ ID NO: 5 comprises 108 bases of 5′-TAACATGTATGATAATGAGCCCAGTACTGCTACTATCAAGAATGAT CTTCGAGATCGTTATCAAGTTTTAAGGAAATTCAGTTCAACAGTCAC AGGGGGTCAATATGC-3′.

Preferably, SEQ ID NO: 5 has other bases added at 5′-end to form SEQ ID NO: 2; more preferably, SEQ ID NO: 5 has other bases added both at 5′-end and 3′-end to form SEQ ID NO: 1.

Preferably, the strength of SEQ ID NO: 5 is greater than SEQ ID NO: 2 or SEQ ID NO: 1 about 1.5 fold.

Another aspect of the present invention relates to a vector comprising an isolated promoter sequence of claim 1.

Preferably, the vector is pGlow-TOPO vector; more preferably, the vector comprises more than 1 isolated promoter sequences of claim 1.

Preferably, the vector comprises 2 isolated promoter sequences of claim 1 with same direction.

Another aspect of the present invention relates to a method for DNA replication, comprising the E. coli/M13 phage cloning system, wherein at least one promoter contains the promoter sequence as claimed in claim 1.

Other objectives, advantages and novel features of the invention will become more apparent from the following detailed description when taken in conjunction with the accompanying drawings.

### BRIEF DESCRIPTION OF THE DRAWINGS

FIG. 1 is the nucleotide sequence of AV3 promoter of AYVV-PD located in nt 615-889 of geminivirus;

FIG. 2 is a genomic map of AYVV-PD depicting AV3 promoter region;

FIG. 3 is a scheme showing the step for producing a mutant AV3 promoter of AYVV-PD by Erase-a-Base system;

FIG. 4 is a series of different fragments of promoter sequences prepared by Erase-a-Base system;

FIG. 5 is a chart showing the amount of the green fluorescence proteins, indicated by relative fluorescent intensities, obtained from different mutants as measured by FLx800™ Multi-Detection Microplate Reader;

FIG. 6 is a chart showing the relative fluorescent intensities of green fluorescence proteins observed from mutants with different promoter repeats as measured by FLx800™ Multi-Detection Microplate Reader;

FIG. 7 is a map showing the direction of the promoter in a recombinant plasmid;

FIG. 8 is a chart showing the analysis of the function of AV3 promoter;

FIG. 9A is the relative position of rep promoter;

FIG. 9B is a chart showing the relative fluorescent intensities of Rep promoter and AV3 promoter as measured by FLx800™ Multi-Detection Microplate Reader;

FIG. 10A is a scheme showing the construction of rrnB P1 promoter by megaprimer; and

FIG. 10B is a chart showing the relative fluorescent intensities of rrnB P1 promoter and AV3 promoter as measured by FLx800™ Multi-Detection Microplate Reader.

### DETAILED DESCRIPTION OF THE INVENTION

The term “isolated” means substantially separated or purified away from contaminating sequences in the cell or organism in which the nucleic acid naturally occurs and includes nucleic acids purified by standard purification techniques as well as nucleic acids prepared by recombinant technology and those chemically synthesized.

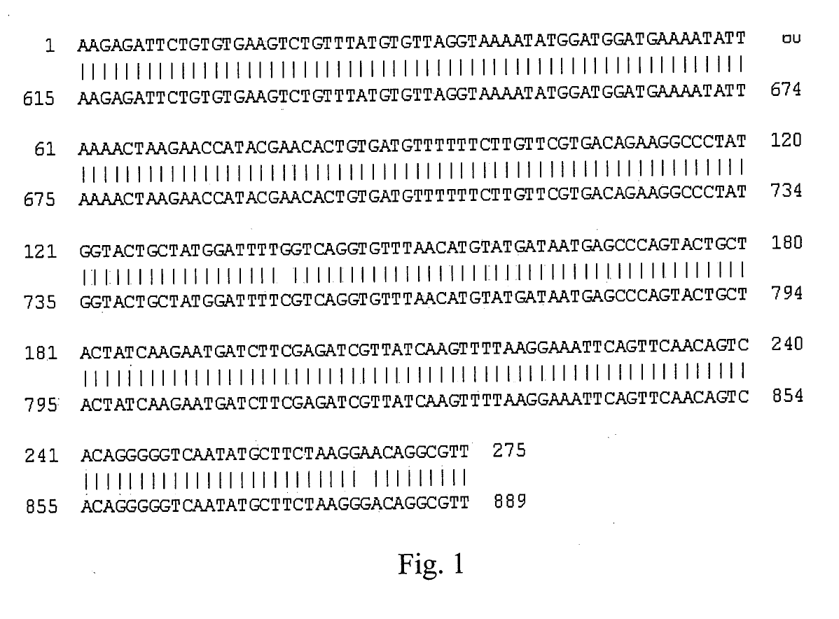

The term “variant” as used herein refers to a DNA molecule wherein the nucleotide sequence is substantially identical to the nucleotide sequence set out in FIG. 1. The variant may be arrived at by modification of the nucleotide sequence of the DNA molecule by such modifications as insertion, substitution or deletion of one or more nucleic acids, such modifications comprising neutral mutations which do not affect the functioning of the DNA molecule.

The term “substantial identity sequences” means that two nucleotide sequences, when optimally aligned, share at least 60 percent sequence identity, preferably at least 80 percent sequence identity, more preferably at least 90 percent sequence identity and most preferably at least 95 percent sequence identity or more.

The term “DNA construct” means a construct incorporating the nucleic acid molecule of the present invention, or a fractional fragment, neutral mutation or homolog thereof in a position whereby a heterologous coding sequence is under the control of and operably linked to the promoter sequence of the invention and is capable of expression in a host cell.

A fragment of a nucleic acid molecule according to the present invention is a portion of the nucleic acid that is less than full length and comprises at least a minimum length capable of hybridizing specifically with a nucleic acid molecule according to the present invention (or a sequence complementary thereto) under stringent conditions as defined below. A fragment according to the present invention has at least one of the biological activities of the nucleic acid or polypeptide of the present invention.

The term “probe” comprises an isolated nucleic acid attached to a detectable label or reporter molecule well known in the art. Typical labels include radioactive isotopes, ligands, chemiluminescent agents, and enzymes.

The term “primers” are short nucleic acids, preferably DNA oligonucleotides 15 nucleotides or more in length, which are annealed to a complementary target DNA strand by nucleic acid hybridization to form a hybrid between the primer and the target DNA strand, then extended along the target DNA strand by a polymerase, preferably a DNA polymerase. Primer pairs can be used for amplification of a nucleic acid sequence, e.g., by the polymerase chain reaction (PCR) or other nucleic acid amplification methods well known in the art. PCR-primer pairs can be derived from the sequence of a nucleic acid according to the present invention, for example, by using computer programs intended for that purpose such as Primer.

Probes or primers can be free in solution or covalently or noncovalently attached to a solid support by standard means.

The term “operably linked” means a first nucleic acid sequence linked to a second nucleic acid sequence when the first nucleic acid sequence is placed in a functional relationship with the second nucleic acid sequence. For instance, a promoter sequence of the present invention is operably linked to a coding sequence of a heterologous gene if the promoter affects the transcription or expression of the coding sequence.

The term “recombinant” nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, eg, by genetic engineering techniques.

The term “expression” refers to the transcription and stable accumulation of sense (mRNA) or functional RNA. Expression may also refer to the production of protein.

The term “vector” refers to a replicon, such as plasmid, phage, cosmid, or virus to which another nucleic acid segment may be operably inserted so as to bring about the replication or expression of the segment.

The term “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

Regarding the amplification of a target nucleic acid sequence (e.g., by PCR) using a particular amplification primer pair, stringent conditions are conditions that permit the primer pair to hybridize only to the target nucleic acid sequence to which a primer having the corresponding wild type sequence (or its complement) would bind.

Nucleic acid hybridization is affected by such conditions as salt concentration, temperature, or organic solvents, in addition to the base composition, length of the complementary strands, and the number of nucleotide base mismatches between the hybridizing nucleic acids, as will be readily appreciated by those skilled in the art.

In addition to eukaryotic cells, geminiviruses could replicate in a prokaryotic cell, although the activities of known geminivirus promoters which have been expressed in the prokaryotic cell are low. The present invention relates to providing a novel and active promoter which expresses foreign genes (containing DNA, RNA and proteins) and providing a way for further understanding the life cycle of geminiviruses for applications in viral disease control.

The method for screening a novel promoter, comprising (1) searching prokaryotic promoter sequence in the constructed AYVV-PD construct and making sure about its minimum length by promoter trapping vector-pGlow-TOPO; (2) a green fluorescent protein (GFP) was taken as a reporter gene to screen the relative strength and to understand the characteristics of isolated promoters; (3) further searching for other unknown genes which relates to replication of prokaryotic cells.

The present invention contemplates a promoter sequence operably linked to a gene of interest.

### EXAMPLES

### Example 1

### Promoter Screening

1.1 Materials

The virus used in the present invention was a Ping-Dong isolate of Ageratum Yellow Vein Virus (AYVV), designated AYVV-PD. The AYVV-PD genome was constructed into a pUC119 vector and sequenced. The construct was named pAger16. The full-length of AYVV-PD was subcloned into a M13 vector and sequenced. The new construct was named Ager16/MP18.

1.2 Random Polymerase Chain Reaction (Random PCR)

Random PCR was performed according to Mullis et al., (1986) and the random primer was designed relative to pGlow-TOPO vector. The random primer was named promoter-trap (SEQ ID NO:6) (Table 1). The primer contains an in-frame ATG start codon for the readingframe of a green fluorescent protein (GFP) sequence on a pGlow-TOPO vector, and provides a ribosome biding site, AGGA. The reaction solution (20 μl) for random PCR comprised 1 μl of AYVV-PD full-length DNA, 1 μl of promoter-trap primer (SEQ ID NO:6) (10 pmole/μl), 2 μl of 10×PCR buffer, 1 μl of 2.5 mM dNTPs, and 0.5 μl (5 U/μl) Taq polymerase. The PCR machine was Gene Amp® PCR System 2400 (Perkin Elmer, PE). The reaction condition was, 94° C. for 5 minutes for DNA denature, then the following were operated for 35 cycle at 94° C. for 30 seconds, 37° C. for 30 minutes, 72° C. for 1 minutes; 72° C. for 1 minutes, and then the reaction was stopped at 25° C. After the reaction was finished, 1 μl of the reaction product was run on 1% agarose and stained by ethidium bromide (EtBr).

TABLE 1
The Primer used in the present invention

| | |
|---|---|
| Primer name | |
| (SEQ ID NO) | Oligonucleotide sequence (5′ → 3′) |

For promoter trapping

| | |
|---|---|
| Promoter trap | CCATATCTATATCTCCTTNNNNN |
| (SEQ ID NO: 6) | |

For inverse PCR

| | |
|---|---|
| G764-783 F | TAACATGTATGATAATGAGC |
| (SEQ ID NO: 7) | |
| G784-803 F | CCAGTACTGCTACTATCAAG |
| (SEQ ID NO: 8) | |
| G795-815 F | ACTATCAAGATGATCTTCGAG |
| (SEQ ID NO: 9) | |
| Inverse R | AAGCTTAAGTTTAAACGCTAGC |
| (SEQ ID NO: 10) | |

For construction of two & three copies promoter

| | |
|---|---|
| 743-871 Fl | TATGGATTTTGGTCAGG |
| (SEQ ID NO: 11) | |
| 743-871 R1 | GTAAGCTTGCATATTGACCC |
| (SEQ ID NO: 12) | |
| 743-871 F2 | GCAAGCTTTATGGATTTTGG |
| (SEQ ID NO: 13) | |
| 743-871 R2 | TAGGATCCGCATATTGACCC |
| (SEQ ID NO: 14) | |
| 743-871 F3 | GCGGATCCTATGGATTTTGG |
| (SEQ ID NO: 15) | |
| 743-871 R3 | CCATATCTATATCTCCTTGCATATTGACCC |
| (SEQ ID NO: 16) | |

For determination of the polarity of promoter

| | |
|---|---|
| C747-871 F | GCATATTGACCCCCCTG |
| (SEQ ID NO: 17) | |
| C747-871 R | CCATATCTATATCTCCTTTATCGATTTTGGTC |
| (SEQ ID NO: 18) | |

For determination of the native ribosome
binding site

| | |
|---|---|
| no rbs 833 F | AAGGGCAATTCTGCAGATC |
| (SEQ ID NO: 19) | |
| no rbs 833 R | TAAAACTTGATAACGATCT |
| (SEQ ID NO: 20) | |

For Northern blot probe preparation

| | |
|---|---|
| V963-949 | GCAAACAACAGATGG |
| (SEQ ID NO: 21) | |

For construction of the rep promoter

| | |
|---|---|
| Rep pro F | AATTGCGAGCACGAATTACTG |
| (SEQ ID NO: 22) | |
| Rep pro R | CCATATCTATATCTCCTTTTTGACTAAGTCA |
| (SEQ ID NO: 23) | ATTGG |

For construction of the rrnB P1 promoter

| | |
|---|---|
| rrnB P1-F | CCTCTTGTCAGGCCGGAATAACTCCCTATA |
| (SEQ ID NO: 24) | ATGCGC |
| rrnB P1 R1 | TTATCCGCTCACAATTCCCTGGTGGCGCATT |
| (SEQ ID NO: 25) | ATAGG |
| rrnB P1 R2 | AACAAAATTATTTCTAGAGGGGAATTGTTAT |
| (SEQ ID NO: 26) | CCGCTC |
| rrnB P1 R3 | CCATATCTATATCTCCTTTTAAAGTTAAACA |
| (SEQ ID NO: 27) | AAATTATT |

1.3 Construction of the Random PCR Product into a pGlow-TOPO Vector

TOPO® Reporter kit (Invitrogen) was used. The provided pGlow-TOPO vector was cut at nt 116-117 and became a linear pGlow-TOPO vector. Each 3′ sticky-end of linear pGlow-TOPO vector was a thymidine (T) base which was easy for selecting PCR product both with A sticky-end and topoisomerase I activity for covalent binding to the vector. After mixing the desired PCR product with the linear vector for 5 minutes at room temperature, the ligation reaction was completed.

pGlow-TOPO vector was a promoter trapping vector. By taking the cycle 3 green fluorescent protein (Cycle 3 GFP) gene as a reporter gene, the inserted PCR product generated using the primer “Promoter trap” (SEQ ID NO:6) could be checked for the presence of any prokaryotic promoter sequence. After transferred into E. coli cells, if the inserted PCR product contains prokaryotic promoter sequence, the promoter sequences could be recognized by RNA polymerase, which transcribes the GFP gene to show green fluorescence. The colonies expressing GFP activities were checked by portable UV lamp (long wavelength, 365 nm).

1.5 Competent Cells Preparation

E. coli TOP 10 cells were cultured in 1.5 ml Luria Bertani medium (LB medium, per liter containing Tryptone 10 g, yeast extract 5 g, NaCl 5 g, pH 7.5). After culturing at 37° C. for 1.5 hr, the culture medium was divided into two centrifugal tubes and centrifuged at 5,000 g for 5 minutes. The supernatants were removed and the pellets were put on ice. Then the pellets were resuspended in 10 ml 0.1 M CaCl₂ individual and put on ice for 30 minutes. After 30 minutes centrifuged at 5,000 g for 5 minutes, and remove the supernatants again. The pellets were resuspended in 1 ml 0.1 M CaCl₂ and put on ice for 1 hr until use. The method for keeping the competent cells is described as follows: one-third volume of 60% glycerol was added into the competent cells to give the final concentration of glycerol of 15%. The well-mixed mixtures were divided into 100 μl aliquots into each eppendorf tube, quick-frozen in liquid nitrogen, and preserved at −80° C.

1.6 Transformation

After ligation, 5 μl recombinant plasmid and 100 μl competent cells were mixed well to form a mixture and put on ice for 30 minutes. Then the mixture was heat shock at 42° C. for 40 seconds and put on ice for 2 minutes immediately. 250 μl LB medium was added into the mixture and then shaking cultured at 37° C. for 40 minutes to obtain a bacterial broth. The bacterial broth was cultured on LA plates with 100 μg/ml Ampicillin. After culture for 8-12 hours at 37° C., desired clones were selected.

1.7 Small Scale Plasmid DNA Purification

A desired clone was selected and cultured in 1.5 ml LB medium with 100 μg/ml Ampicillin. After cultured for 12-14 hours at 37° C., the bacterial broth was centrifuged at 14,000 rpm for 1 minute and the supernatant was removed and 100 μl solution I (50 mM glucose, 10 mM EDTA pH 8.0, 25 mM Tris pH 8.0) was added. After thoughtfully mixing, 200 μl solution II was added (0.2 N NaOH, 1% sodium dodecyl sulfate) smoothly to mix well. The mixture was put on ice for 5 minutes and 150 μl solution III (5 M potassium acetate, pH 4.8) was added smoothly and put on ice for 15 minutes. Then the mixture was centrifuged at 14,000 rpm for 15 minutes.

The supernatant was transferred into a new eppendorf and 300 μl PCI was added and mix well, then centrifuged at 14,000 rpm for 5 minutes. After centrifugation, the supernatant was removed into another new eppendorf and 0.1× volume of 3 M Sodium Acetate (NaOAc) and 2.5× volume of 95% ethanol were added. After precipitating at −80° C. for more than 15 minutes, centrifuging 14,000 rpm for 15 minutes, a precipitate was obtained. The precipitate was washed by 70% ethanol and centrifuged at 14,000 rpm for 5 minutes. Finally, the pellet was dried by vacuum and re-dissolved in 40 μl dH₂O. 1 μl plasmid DNA was separated by electrophoresis on 1% agar gel.

1.8 NheI Restriction Enzyme Reaction

To further examine the size of inserted fragment, NheI restriction enzyme cut at nt 50 and nt 144 at the vector. The reaction solution was prepared as follow: 1 μl plasmid DNA, 1 μl of 10× buffer (50 mM NaCl, 10 mM tris-HCl, 10 mM MgCl₂, 1 mM dithiothreitol, pH 7.9), 1 μl of 100 μg/ml BSA, 0.5 μl NheI restriction enzyme (New England Biolabs) and 6.5 μl dH₂O. Total volume was about 10 μl. After mixing well and culturing at 37° C. for 2 hours, 3 μl of the solution was taken for electrophoresis on 2% agar gel for determining the size of the fragments.

1.9 Sequence Analysis

According to the principle of cycle sequencing, SequiTherm EXCEL™ II kit (Epicentre) was used and reactions were performed with LI-COR 4200 automatic DNA Sequencer for sequence analysis. Reaction samples were prepared described as followed. 2 μl DNA template (1 μg/μl), 1 μl IRD 700 T7 promoter primer (3.2 pmol/μl), 7.2 μl 3.5× buffer, 0.5 μl Excel II DNA polymerase and 6.3 μl dH₂O, and total volume was 17 μl. The reaction solution was mixed well and divided into 4 tubes (4 μl/tube), each of which contained 2 μl of one of the four different dideoxy ribonucleotides (ddNTP) respectively, then DNA amplified by PCR reaction. Condition for PCR reaction is described as follow: 94° C. for 5 minutes for DNA denaturing, then the following were operated for 30 cycle at 94° C. for 30 seconds, 50° C. for 15 minutes, 72° C. for 1 minutes, and finally the reaction stopped at 25° C.

Then 3 μl/tube loading dye (98% formamide, 2 mM EDTA, pH 8.0) was added into each tube and heated at 95° C. for 5 minutes for DNA denaturing and put on ice immediately until used.

LI-COR 4200 automatic Sequencer was used for DNA sequencing. The results of the sequenced DNA were analyzed by BLAST software on NCBI web. Results showed that all DNA fragments were from the same region of AYVV-PD, and the fragment representing nt 615-889 of virion-sense showed prokaryotic promoter activity. Therefore, the above fragment was named pGP615-889, and the promoter was named AV3 promoter.

Results

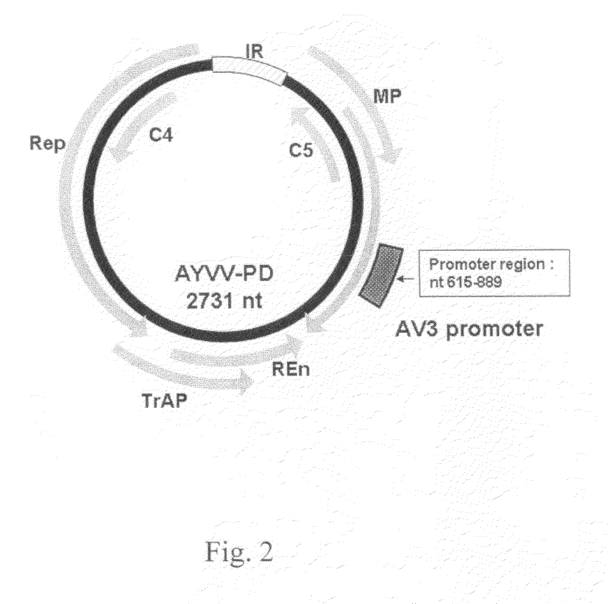

According to the above procedure, we found that all selected fragments were located at the same region of AYVV-PD genome and the smallest fragment was located at nt 615-889 (SEQ ID NO: 1) (FIG. 1) on AYVV-PD genome. The recombinant construct harboring this fragment was named pGP615-889. There are two known open reading frames in the virion-sense direction of geminivirus genome, one is AV1 movement protein which is located at nt 134-484 of the AYVV-PD, and the other is AV2 coat protein which is located at nt 294-1067 of the AYVV-PD genome sequences. The novel promoter is different from the above two fragments, and hence was named AV3 promoter (FIG. 2).

### Example 2

### The Smallest Functional Length of AV3 Promoter of AYVV-PD

2.1 Erase-a-Base® System

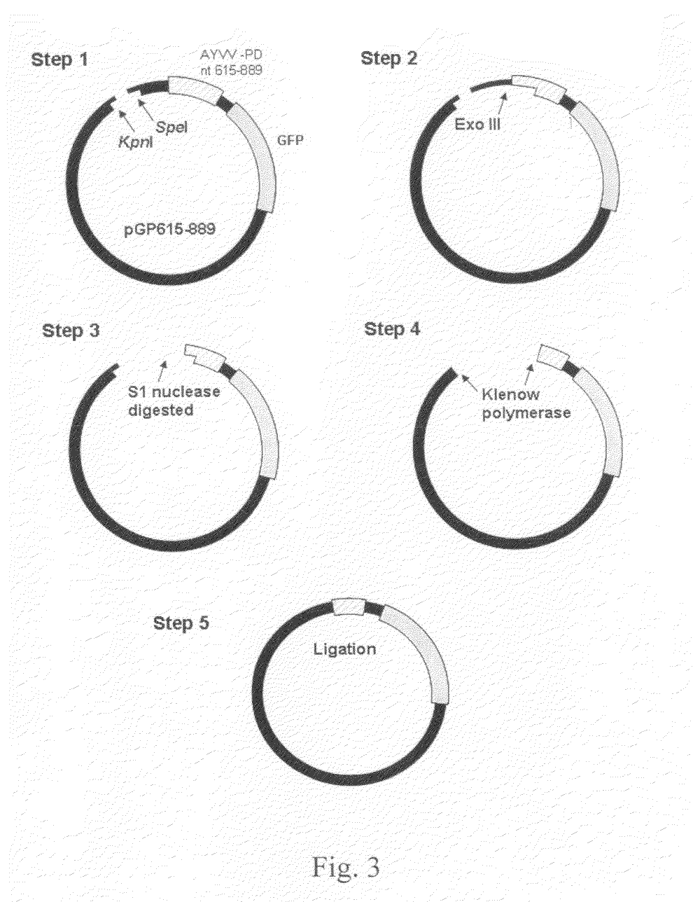

(1) With reference to FIG. 3, Erase-a-Base system (Promega) was used for one-way deletion. First, two restriction enzyme cutting sites, KpnI and SpeI, were found at the up-strand of pGP615-889 promoter sequence of the plasmid (inserted at nt 116-117 of pGlow-TOPO vector) at nt 76 and nt 90, respectively. Second, the plasmids were cut by the two restriction enzymes to generate 3′-sticky end (cut by KpnI) and 5′-sticky end (cut by SpeI), and analyzed by 1% agarose gel.

(2) After quantifying the concentration of linear DNA plasmids to reach 1.25 μg, 1.5 μl of 10× exonuclease III buffer (660 mM Tris-HCl pH 8.0, 6.6 mM MgCl₂) and dH₂O was added to make total volume reach 15 μl, then kept the solution at 11° C.

(3) 7.5 μl S1 nuclease mix was respectively added into 6 new eppendorfs, and each tube contained 2.4 U S1 nuclease and 1.08 μl S1 nuclease buffer (0.3 M potassium acetate, pH 4.6, 2.25 M NaCl, 16.9 mM ZnSO₄, 45% glycerol), and dH₂O was added to make total volume reach 7.5 μl, then the S1 nuclease mix was put on ice for use.

(4) At 0 minute, 2.5 μl of reaction product was mixed with S1 nuclease mix as control. Then, 100 U exonuclease III was added into reaction product and 2.5 μl of reaction product was taken out and added into S1 nuclease mix every 1 minute. After all the reaction product was taken off, all eppendorfs were put in room temperature for 30 minutes.

(5) 1 μl of S1 nuclease stop buffer (0.3 M Tris-base, 0.05M EDTA) was added at 70° C. for 10 minutes to stop the S1 nuclease activity. 0.3× volume of 7.5 M ammonium acetate and 2× volume of 95% ethanol were added and put on −20° C. for more than 15 minutes. After centrifuging 14,000 rpm for 15 minutes, the supernatant was removed and pellet was washed by 70% ethanol and further centrifuging at 14,000 rpm for 5 minutes. Pellet was dried by vacuum drier and re-dissolved by 11 μl TE buffer. 2 μl was taken for electrophoresis.

(6) 1 μl Klenow mix was added into each tube, the Klenow mix comprises 1 μl of 1× Klenow buffer (20 mM Tris-HCl pH 8.0, 100 mM MgCl₂), and 0.17 U Klenow DNA polymerase. When pre-heat at 37° C. for 3 minutes, 1 μl dNTP mix was added and reaction was continued for 5 minutes, then the tubes were transferred to a heater with 65° C. for 10 minutes to inactivate Klenow DNA polymerase.

(7) The tubes were taken out from the heater and cool down. 40 μl of ligase mix was added into each tube. The ligase mix comprises 4 μl of 10× ligase buffer (500 mM Tris-HCl pH 7.6, 100 mM MgCl₂, 10 mM ATP), 4 μl of 50% PEG, 0.4 μl of 100 mM DTT, 0.2 U T4 DNA ligase and 31.6 μl dH₂O. Each component were mixed well and put at room temperature for 1 hour, and proceed to transformation.

(8) NheI restriction enzyme was used for promoter length analysis.

2.2 Inverse PCR Reaction

PCR primer pairs were designed as: G764-783F (SEQ ID NO:7), G784-803F (SEQ ID NO:8), G795-815F (SEQ ID NO:9) and Inverse R (SEQ ID NO:10) (Table 1). The three forward primers paired with the reverse primer (Inverse R) (SEQ ID NO:10) for operating PCR. Total PCR solution (20 μl) comprises 1 μl pGP615-889 DNA, 1 μl forward primer (10 pmol/μl), 1 μl reverse primer (10 pmol/μl), 2 μl of 10×PCR buffer, 1 μl of 2.5 mM dNTPs and 0.5 μl Taq polymerase (5 U/μl). The PCR condition was: 94° C. for 5 minutes; 94° C. for 30 seconds, 50° C. for 30 seconds, 72° C. for 5 minutes, continued for 30 cycles, 72° C. for 1 minutes and then the reaction was stopped at 25° C.

Then the PCR products were separated by 1% agarose gel and stained by EtBr. After electrophoresis, 0.1× volume of 3M NaOAc and 3× volume of 95% ethanol were mixed with 200 μl of desired products to precipitated the desired PCR products. The precipitate was re-dissolved in 9.1 μl dH₂O for ligation.

2.3 Ligation

1 μl of the precipitate obtained from Example 2.2 was mixed with 10×T4 DNA ligation buffer (250 mM Tris-HCl pH7.6, 50 mM MgCl₂, 5 mM ATP, 5 mM DTT, 25% (w/v) polyethylene glycol-8000), 0.4 μl of 25 mM ATP and 0.5 μl T4 DNA ligase at room temperature for 1 hour. Then NheI restriction enzyme was used for selecting and further checking the sequence.

Results

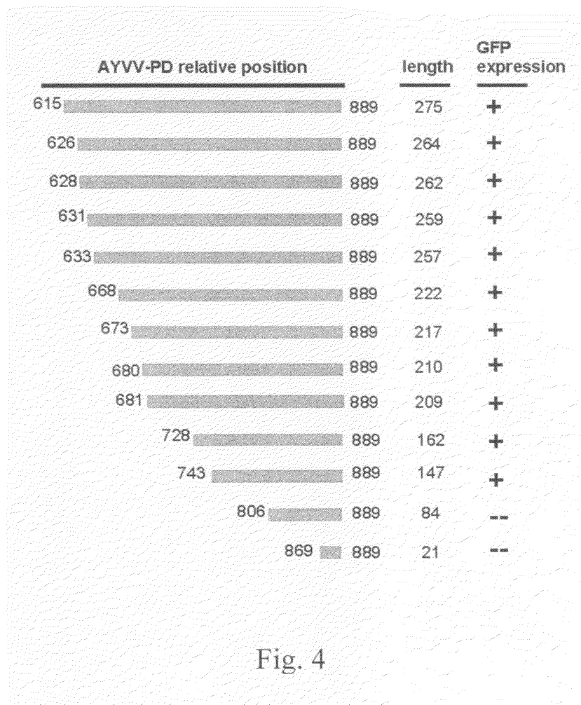

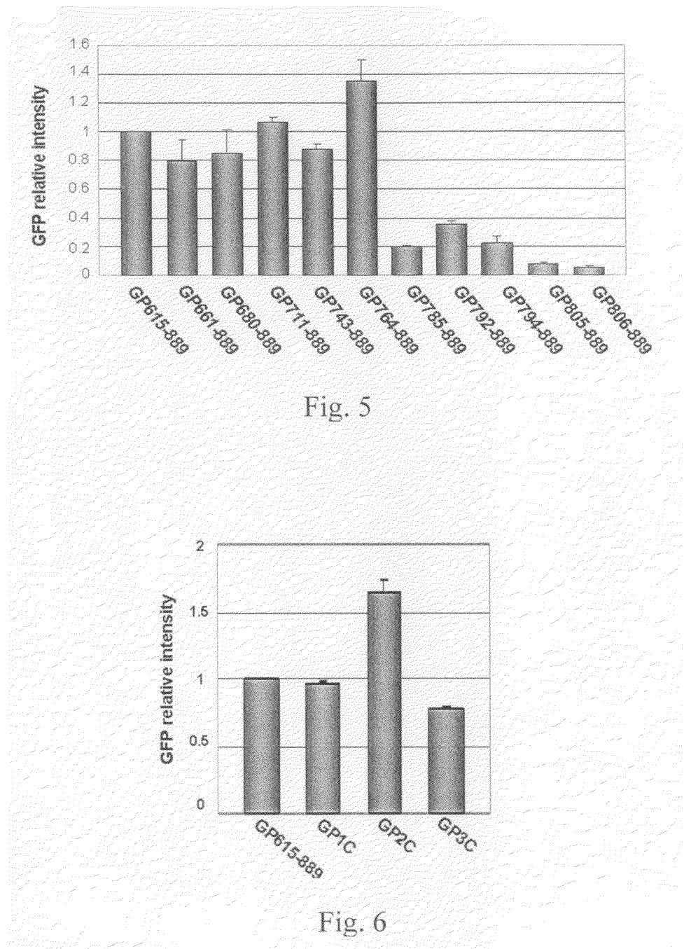

After digesting by NheI restriction enzyme, fragments with lengths greater than 100 by were selected and checked whether such fragments express GFP or not (FIG. 4). It was shown when 5′-end was digest until nt 743 of the pGP615-889 recombinant construct, the sequence still showed promoter activity, however, when 5′-end was digest until nt 806 of the pGP615-889 recombinant construct, the sequence could not shown the promoter activity. To further understand the precise site of the core promoter at nt 743-806 region, the present invention checked the region by inverse PCR reaction and restriction enzyme. Furthermore, the relative intensity of the different length of promoter sequences were checked by FLx800 Multi-Detection Microplate Reader (FIG. 5). Results showed when 5′-end was digest between nt 615 to nt 743 (SEQ ID NO: 2), the relative intensity was approximately the same. However, when 5′-end was digested until nt 764 (SEQ ID NO: 3), the relative intensity was raise about 1.5 fold. GFP expression amount also rise when detected by Western Blotting. When 5′-end was digested to nt 785 (SEQ ID NO: 4), the relative intensity was reduced to 0.2 fold. When 3′-end was digested to nt 871 and detected by Microplate Reader, the result revealed nt 764-871 still showed promoter activity. Therefore, the smallest length of the promoter sequence is at nt 764-871 (SEQ ID NO: 5) in present invention provisionally.

### Example 3

### Construct with 2-Repeat Promoter Sequences or 3-Repeat Promoter Sequences

3.1 PCR

The primer pairs used in PCR were designed according the AYVV-PD AV3 promoter sequence, and a total of 6 primers (3 pairs) were synthesized, which comprise: 743-781 F1 (SEQ ID NO:11), 743-781 R1 (SEQ ID NO:12), 743-871 F2 (SEQ ID NO:13), 743-871 R2 (SEQ ID NO:14), 743-871 F3 (SEQ ID NO:15), 743-871 R3 (SEQ ID NO:16) (table 1). The primers labeled with R1 (SEQ ID NO:12) and F2 (SEQ ID NO:13) were designed with HindIII cutting site, the primers labeled with R2 (SEQ ID NO:14) and F3 (SEQ ID NO:15) were designed with BamHI cutting site, and the primer labeled with R3 (SEQ ID NO:16) was designed with ATG sequence which is in the same reading frame as the initiation site (ATG) of GFP on pGlow-TOPO vector, and also comprise RBS sequence.

The 3 groups of primer pairs were operated respectively by PCR; the reaction solution was the same as the previous one. The reaction condition was: 94° C. for 5 minutes; 94° C. for 30 seconds, 50° C. for 30 seconds, 72° C. for 30 seconds, continued for 35 cycles; 72° C. for 30 seconds and then the reaction was stopped at 25° C. The PCR products were precipitated by ethanol.

3.2 HindIII and BamHI Restriction Enzyme Reaction

The PCR products obtained from example 3.1 were digested by HindIII and BamHI (TaKaRa) respectively at 37° C. (HindIII) and 30° C. (BamHI) for 2 hours. The products reacted with restriction enzymes were precipitated by ethanol and analyzed by 1% agarose. 2 or 3 fragments of the cutting products were ligased with pGlow-TOPO vector and checked by NheI restriction enzyme to understand the size, the orientation and sequences were check by nucleic acids sequencing.

Results

To further understand whether the AV3 promoter show additivity, the relative intensity of the promoter constructs were checked by Microplate Reader (FIG. 6), and GFP expressed by GP615-889 was taken as 1. Results showed that when 2-repeat promoter sequences were constructed on the vector, the relative intensity was 1.7; and when 3-repeat promoter sequences were constructed on the vector, the relative intensity was 0.7. Therefore, the promoter sequence shows additivity in 2-repeat construction.

### Example 4

### Detection of GFP Expression

4.1 Western Blot Assay

The various constructs obtained according to the present invention were transferred into E. coli TOP10 and cultured at 1.5 ml of LB medium with 100 μg/ml Ampicillin. After culturing at 37° C. for 12-14 hours, absorption value was measured at OD₆₀₀, the OD₆₀₀ was adjusted to almost the same. Then, 16 μl of culture broth was mixed with 4 μl of 5×SDS loading dye and heated at 100° C. for more than 15 minutes. 5 μl of each product were taken and separated by 12.5% SDS PAGE (190V, 50 minutes).

After running on SDS PAGE, the gel was rinse in a transfer buffer (25 mM Tris, 190 mM glycine, 20% methanol) for 5 minutes. And as the same time, prepare the PVDF membrane and 2 sheets of 3MM filters. The PVDF membrane was treated with methanol for 3-5 seconds and then washed by dH₂O until the membrane recover water completely, and rinse in transfer buffer for 15 minutes. Until all the preparation had finished, proceeds western blotting (90 V, 60 minutes). The membrane was detected by antibody after transfer. Protein detection techniques are commonly known and used by those in the art.

4.2 FLx800™ Multi-Detection Microplate Reader Detection

The various constructs were transferred in to E. coli TOP 10 cells and cultured in 2 ml of LB medium with 100 μg/ml at 37° C. for 12-14 hours. The cultured broth were divided into 96 well plate, each well contained 90 μl cultured broth and operate 3 repeat. The concentration of each well was measured at OD₆₀₀. Furthermore, different samples were also put in 96 well plate with black bottom for 3 repeats, and the GFP expression were also detected at 400 nm (excitation), 508 nm (emission). The detection software is Gen5 Data Analysis Software.

The obtained OD values and GFP expression levels were respectively calculated to obtain an average value. The following formulation was performed to get GFP expression level in arbitrary unit.

The average value of GFP expression level/the average value of OD value=GFP expression level in per unit

GFP expression level in pGP615-889 was assigned as 1, and then used as the standard to calculate the relative values of the other samples. Such method was also used in the following example 5.2.

### Example 5

### Orientation of the Promoter Sequence

5.1 PCR

The used primers were designed according to the AYVV-PD AV3 promoter sequence for inserting the promoter at reverse orientation. The primer pair was C747-871 F (SEQ ID NO:17) and C747-871 R(SEQ ID NO:18) (table 1). The primer pair also carried ATG sequence which was in the same reading frame as the initial sequence of GFP gene on the pGlow-TOPO vector. The reaction condition was the same as described previously.

5.2 Detection of GFP Expression

As described in Example 4.

5.3 Inverse PCR

To further understand the ribosome binding site (RBS) of AYVV-PD AV3 promoter, the used primer pair were no rbs 833 F (SEQ ID NO:19) and no rbs 833 R (SEQ ID NO:20) (table 1). The PCR condition was the same as described previously. The PCR products were precipitated by ethanol and follow operated ligation and transformation.

5.4 Northern Blot Assay

5.4.1 Probe Preparation

To detect mRNA of GFP, a reverse primer, V963-949 (SEQ ID NO:21) (nt 949-963), was designed at the down-strand of GFP sequence (nt 140-859) on pGlow-TOPO vector. Such primer was used for preparing detected probe. Reaction condition: 1 μl of pGP615-889 DNA, 2.9 μl of 3.5× buffer, 1 μl of V963-949 primer (SEQ ID NO:21), 1 μl of 10×DIG-DNA mix and 0.5 μl of Excel II DNA polymerase, then dH₂O was added to make the total volume reach 10 μl. The reaction machine was Gene Amp® PCR System 2400 (Perkin Elmer, PE). The reaction condition was: 94° C. for 5 minutes; 94° C. for 30 seconds, 55° C. for 15 seconds, 72° C. for 45 seconds continued for 50 cycles; and then the reaction was stopped at 4° C. The PCR products were precipitated by ethanol for use.

5.4.2 Sample Preparation

A single clone was selected from the culture plate and cultured in 1.5 ml of LB medium containing 100 μg/ml Ampicillin at 37° C. for 12-14 hours. Then the culture medium was centrifuged at 14,000 rpm for 1 minute to obtain bacterial pellet. A hot phenol solution containing 400 μl of phenol and 250 μl lysis buffer (0.4 M NaCl, 40 mM EDTA, 1% β-mercapto-ethanol, 1% sodium dodecyl sulfate, 20 mM Tris pH7.4) was added in a new eppendorf and pre-heated on a heater at 90° C. for more than 10 minutes. Then 250 μl of resuspension buffer (10 mM KCl, 5 mM MgCl₂, 10 mM Tris-HCl pH 7.4) was mixed with the bacterial pellet to obtain a bacterial suspension. The bacterial suspension then was mixed with hot phenol.

The bacterial suspension mixed with hot phenol was centrifuged at 14,000 rpm for 5 minutes and a first supernatant was collected. Another 300 μl of phenol was mixed with the first supernatant and further centrifuged at 14,000 rpm for 5 minutes to obtain a second supernatant. The second supernatant was mixed with 300 μl of PCI and then centrifuged at 14,000 rpm for 5 minutes and repeat for twice. Finally, the collected supernatant was precipitated by ethanol and dried by vacuum to obtain a precipitated pellet. The precipitated pellet was re-dissolved by 25 μl of gel loading buffer II (95% formamide, 18 mM EDTA, 0.025% SDS per tube, 0.025% xylene cyanol per tube, 0.025% bromophenol blue per tube) and then heat at 100° C. for 10 minutes. The samples were checked by 1% agarose gel and stained by EtBr, electrophoresis condition was 80V for 80 minutes.

5.4.3 Northern Blot Assay

The sample prepared by example 5.4.2 was separated by 1% agarose gel without EtBr and stored in 20×SSC buffer (2 M NaCl, 2M sodium citrate). Another NC membrane was rinsed in 2×SSC buffer for 5 minutes and then stored in 20×SSC buffer for use.

Further Northern blot techniques are commonly known and used by those in the art.

Results

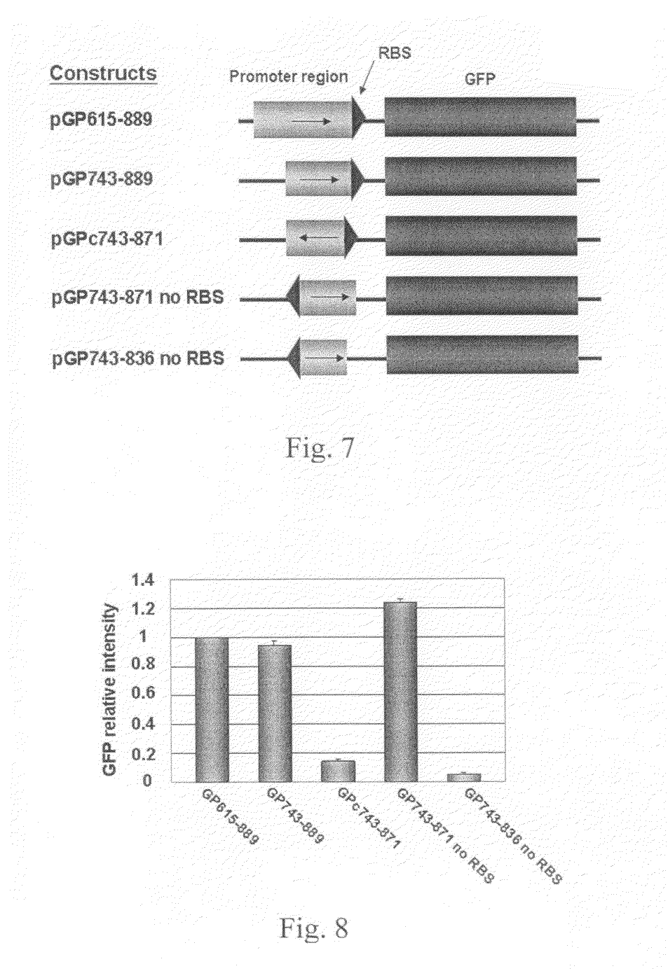

The genome of geminivirus is ambisense, and the promoter sequence located in common region is bi-directional in that it direct the transcription of Rep gene at complementary-sense direction and the transcription of coat protein gene at virion-sense direction. Therefore, we tried to construct AYVV-PD nt 747-871 fragment at reverse direction and also to provide RBS and ATG biding site to examine the active direction of AV3 promoter. With reference to FIGS. 7 and 8, western blot assay and Microplate Reader were also used for analyzing the enhancement ability of promoter.

The desired construct with reverse-insert was named pGPc743-871 and showed no promoter ability.

But the pGP743-871 no RBS (FIG. 7) recombinant plasmid showed promoter activity (FIG. 8), implying that the sequence between nt 743-871 have a native ribosome binding site. Surveying the 3′ end of nt 743-871, it was found that the site at nt 834-837 contain a RBS sequence (AGGA).

In order to understand if there is really a RBS in the promoter sequence, inverse PCR were used to construct a recombinant plasmid without this native RBS. After confirmation by sequencing, the construct was named pGP743-836 no RBS (FIGS. 7 and 8). The promoter activity of this construct was analyzed through Western blot assay, Microplate Reader and Northern blot assay. The results show that this construct cannot express GFP, but the promoter still has function to drive the transcription of GFP mRNA.

### Example 6

### Comparison with Rep Promoter Obtained from Geminivirus

6.1 Rep Promoter Construction

6.1.1 PCR

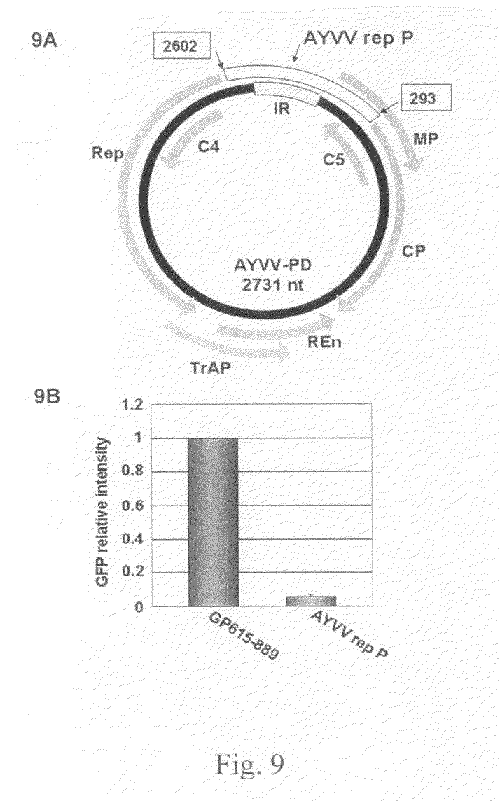

With reference to FIG. 9A, to amplify intergenic region between AYVV-PD Rep gene and coat protein gene (nt 2602-293), and the primer pair used for amplification were Rep pro F (SEQ ID NO:22) and Rep pro R(SEQ ID NO:23). The reverse primer had ATG sequence which was in the same reading frame as initial sequence of GFP gene on the pGlow-TOPO vector, and also had RBS sequence (AGGA). The reaction solution was prepared as described previously. The reaction condition was: 94° C. for 5 minutes; 94° C. for 30 seconds, 53° C. for 30 seconds, 72° C. for 30 seconds continued for 35 cycles; and 72° C. for 30 seconds, then the reaction was stopped at 25° C.

6.2 GFP Expression Detection

Sequence of the constructed plasmid was confirmed and the relative intensity of promoter sequence was checked by FLx800™ Multi-Detection Microplate Reader.

Results

To compare the relative intensity of AV3 promoter and Rep promoter, the Rep promoter sequences were amplified by PCR and constructed in pGlow-TOPO vector. The construct was named pAYVV rep P. With reference to FIG. 9B, results showed that the relative intensity of GP615-889 was about 15-fold greater than that of AYVV rep P.

### Example 7

### Comparison with rrnB P1 Promoter on E. coli

7.1 rrnB P1 Promoter Construct

7.1.1 Mega-Primer PCR

The primers used for amplifying rrnB P1 promoter were rrnB P1-F (SEQ ID NO:24), rrnB P1-R1 (SEQ ID NO:25), rrnB P1-R2 (SEQ ID NO:26) and rrnB P1-R3 (SEQ ID NO:27) (table 1). The reverse primer, rrnB P1-R3 (SEQ ID NO:27), harbors the ATG sequence which is in the same reading frame as initial sequence of GFP gene on the pGlow-TOPO vector, and also provides RBS sequence (AGGA).

The reaction solution was: 1 μl of each primer (100 pmole/μl), 10×PCR buffer, 1 μl of 2.5 mM dNTPs and 0.5 μl of Taq polymerase (5 U/μl), and dH₂O was added to make total volume reach 20 μl. The reaction was performed on Gene Amp® PCR System 2400 (Perkin Elmer, PE), and the condition was: 94° C. for 5 minutes; 94° C. for 30 seconds, 60° C. for 30 seconds, 72° C. for 15 seconds continued for 35 cycles; and 72° C. for 10 seconds, then the reaction was stopped at 25° C.

1 μl of PCR product was used for checked and stained by EtBr. Then the correct PCR product was inserted in pGlow-TOPO vector and transferred into E. coli TOP 10. Desired clones that expressed GFP were selected.

7.2 Comparison of GFP Expression Levels

The relative intensity of rrnB P1 promoter and AYVV-PD AV3 promoter was detected by FLx800™ Mutli-Detection Microplate Reader and Western blotting.

Results

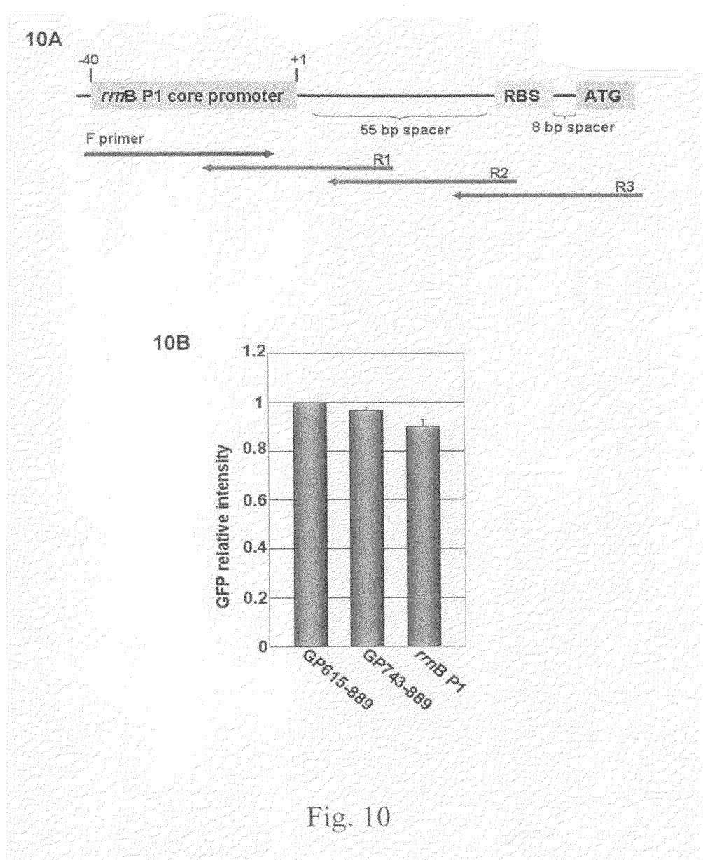

With reference to FIG. 10A, rrnB P1 promoter construction detail was shown. The construct containing rrnB P1 promoter and expressing GFP was named prrnB P1 plasmid. By comparing the GFP expression levels using Western blot and FLx800™ Mutli-Detection Microplate Reader, the relative intensity of rrnB P1promoter was found to be approximately equal to that of AYVV-PD AV3 promoter. If the relative intensity of AYVV-PD AV3 promoter is 1, the relative intensity of rrnB Plpromoter is 0.9.

In conclusion, the characteristics of the present invention are described as follow.

1. AV3 promoter is a novel promoter found in geminivirus. AV3 promoter locates at C-terminus of coat protein gene in geminivirus, such location never shows any promoter activity in previous studies.

2. Although geminiviruses are regarded as eukaryotic viruses which could express their genes in eukaryotic cells, the AV3 promoter is highly active in prokaryotic cells and shows the characteristics of prokaryotic promoter. Therefore, AV3 promoter could be utilized efficiently by prokaryotic RNA polymerase.

3. AV3 promoter is a constitutive promoter and could be highly expressed without inducer. Such method for using AV3 promoter will reduce cost.

4. The activity of AV3 promoter is about 15-fold higher than the known Rep promoter. The activity of AV3 promoter is also higher than the constitutive promoter, rrnB P1 of E. coli, about 11.1% more.

5. The AV3 promoter shows additivity. Two repeated AV3 promoters of the same direction show higher activity than single AV3 promoter for about 1.7 fold.

6. The AV3 promoter could only be expressed in one direction and drive virion-sense gene in one direction. Such way for application is convenient.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An isolated promoter sequence comprising SEQ ID NO: 5 or a substantial identity sequence.

2. The isolated promoter sequence as claimed in claim 1, wherein SEQ ID NO: 5 is isolated from Ageratum yellow vein virus.

3. The isolated promoter sequence as claimed in claim 1, wherein SEQ ID NO: 5 comprises 108 bases of 5′-TAACATGTATGATAATGAGCCCAGTACTGCTACTATCAAGAATGAT CTTCGAGATCGTTATCAAGTTTTAAGGAAATTCAGTTCAACAGTCAC AGGGGGTCAATATGC-3′.

4. The isolated promoter sequence as claimed in claim 1, wherein SEQ ID NO: 5 has other bases added at 5′-end to form SEQ ID NO: 2.

5. The isolated promoter sequence as claimed in claim 1, wherein SEQ ID NO: 5 has other bases added both at 5′-end and 3′-end to form SEQ ID NO: 1.

6. The isolated promoter sequence as claimed in claim 5, wherein the strength of SEQ ID NO: 5 is greater than SEQ ID NO: 2 or SEQ ID NO: 1 about 1.5 fold.

7. A vector comprising an isolated promoter sequence of claim 1.

8. The vector as claimed in claim 7, wherein the vector is pGlow-YOPO vector.

9. The vector as claimed in claim 7, wherein the vector comprises more than 1 isolated promoter sequences of claim 1.

10. The vector as claimed in claim 9, wherein the vector comprises 2 isolated promoter sequences of claim 1 with same direction.

11. A method for gene expression, comprising
providing an expression vector containing a promoter operably linked to a gene of interest, wherein the promoter containing a promoter sequence as claimed in claim 1; and
expressing the gene of interest in the expression vector.
